SEQUENCE LISTING (1 sequence)

SEQ ID NO: 1 - protein, 9 aa, artificial sequence
  synthetic LH-RH analogue
XHWSYXLRX

(19) United States
(12) Patent Application Publication
(10) Pub. No.: US 2004/0248804 A1
(43) Pub. Date: Dec. 9, 2004

(54) Nasal administration of the LH-RH analog Leuprolide

(76) Inventors: Khurshid Iqbal, Berwyn, PA (US); Anthony Fisher, Leicestershire (GB); Peter Watts, Nottingham (GB)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(21) Appl. No.: 10/838,077

(22) Filed: May 3, 2004

Related U.S. Application Data
(60) Provisional application No. 60/467,095, filed on May 1, 2003.

(51) Int. Cl. A61K 38/09

(52) U.S. Cl. 514/12; 514/55

(57) ABSTRACT

The invention provides a composition for a nasal administration that includes leuprolide and one bioadhesive material that is chitosan. Methods of administration of leuprolide to a mammal suffering from a leuprolide modulated condition, such as endometriosis, prostate cancers, hypogonadism, pre-menstrual syndrome, or uterine leiomyomas.

### CROSS-REFERENCE TO RELATED APPLICATIONS

The application claims priority to U.S. provisional patent application Ser. No. 60/467,095, filed May 1, 2003, the contents of which are incorporated herein by reference.

### BACKGROUND OF THE INVENTION

Luteinizing hormone (LH) and follicular stimulating hormone (FSH) are produced by certain gonadotroph cells of the anterior pituitary gland of the mammal. FSH and LH act on the gonads to stimulate the production of steroid hormones (such as progesterone oestradiol in human females; testosterone in males) and to stimulate gamete maturation in male and female mammals. In addition to being necessary for normal reproductive development, abnormal or inconsistent levels of FSH and/or LH have been implicated by researchers in numerous conditions and diseases involving the reproductive organs and/or irregularities in the hormonal levels present in the reproductive cycle.

The production of LH and FSH is regulated in males and in females humans by gonadotropin-releasing hormone (GnRH), sometimes referred to as luteinizing hormone-releasing hormone (LH-RH). LH-RH is believed to be the sole neuropeptide causing the release of LH and FHS. Naturally occurring LH-RH is produced in the hypothalamus in response to neural and/or chemical stimuli and released into the hypophyseal portal circulation. It is believed that LH-RH modulates LH and FHS production by binding to gonadotropin-releasing hormone receptors (“GnRHRs”) that are expressed on the cells of the pituitary gland, ovaries, breast, testis, and prostate, thereby initiating the phosphatidylinositol-Ca⁺ second messenger system.

The therapeutic use of LH-RH or LH-RH analogs in the treatment of various disorders has been proposed and, in some cases, attempted. Among the synthetic analogs of LH-RH is “leuprolide” or “leuprolide acetate.” Clinical trials have demonstrated that continuous therapy with leuprolide produces an initial stimulation of the FSH and LH in the patient, followed by suppression of these hormones, with reduction of gonadal hormones to levels similar to those measured in castrated or post-menopausal individuals. In men, the net effect of administration of leuprolide is a reduction of testosterone levels to castration levels in two to four weeks. In females, both ovarian estrogen and androgen synthesis are inhibited. This reduction or inhibition facilitated by leuprolide has been shown effective in the treatment of numerous disorders, including endometriosis and prostate cancer.

However, the administration of leuprolide to patients in an effective, tolerable and bioavailable manner is problematic. Leuprolide cannot be administered orally because it is destroyed in the environment of the gastrointestinal tract. Thus, leuprolide is conventionally administered by invasive methods such as intravenously, via subcutaneous or intramuscular injection, or by subcutaneous insertion of a device that releases leuprolide over an extended period of time. However, administration by invasive methods is inconvenient (often requiring daily injections and/or frequent office visits), may be expensive because of the equipment required, and may cause the patient to experience pain, discomfort, and/or inflammation or infection at the site of injection or insertion. These disadvantages may result in reduced patient compliance and increased medical costs. Thus, there is a need in the art for non-invasive methods of leuprolide delivery and compositions formulated for such delivery.

### BRIEF SUMMARY OF THE INVENTION

The invention includes a composition for nasal administration that includes leuprolide and one bioadhesive material that is chitosan. The composition may also include an administration vehicle, such as a nasal administration vehicle. The invention also includes compositions for nasal administrations that consist essentially of chitosan, leuprolide, a preservative, and a nasal administration vehicle.

The invention encompasses methods of administration of leuprolide to a mammal subject suffering from a leuprolide modulated condition. The method includes contacting the nasal mucosa of the mammal with the composition of the invention.

Also provided are methods of inhibiting the net production of estrogen in a female mammal or the net production of testosterone in a male mammal. These methods include repeatedly contacting the nasal mucosa of the mammal with a composition of the invention over a time period, such as at least fourteen days.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 shows the leuprolide concentrations (nanograms per milliliter) present in subjects' blood plasma over time. Concentration levels are shown for intravenous, subcutaneous, and nasal administration. The nasal administration was accomplished using the compositions and the methods of the invention.

FIG. 2 shows the maximal plasma concentration (nanograms per milliliter) for each route of administration.

FIG. 3 shows the area under the curve (AUC) of plasma concentration versus time for each route of administration;

FIG. 4 compares the absolute bioavailability of leuprolide administered subcutaneously, and intranasally, as a percent value of leuprolide administered intravenously.

### DETAILED DESCRIPTION OF THE INVENTION

The invention provides compositions and methods for administration of leuprolide to a mammal via the mucosal surfaces of a mammal, specifically the nasal mucosa. Upon administration by the method of the invention, the leuprolide composition is well-tolerated by the patient, is well-absorbed by the system of the patient, and results in plasma leuprolide levels that approximate those observed in a patient following subcutaneous administration. Additionally, because the instant methods of leuprolide delivery are non-invasive, issues of drug degradation in the gastrointestinal tract and of reduced patient compliance are avoided.

A leuprolide delivery composition is included in the invention and is used in the practice of the methods of the invention. The composition includes at least (i) leuprolide and (ii) chitosan. A suitable administration vehicle may be included, such as a nasal administration vehicle.

“Leuprolide” for use in the invention encompasses leuprolide acetate and all derivatives, and salts thereof known or to be developed in the art. Also included are polypeptides of greater than nine residues having as a portion of their primary sequence the sequence:

Xaa-His-Trp-Ser-Tyr-Xaa-Leu-Arg-Xaa [SEQ ID NO: 1],

when “Xaa” at position 1 is a 5-oxo-prolyl residue, “Xaa” at position 6 is a D-leucyl residue, and “Xaa” at position 9 is a prolyl-N-ethyl amide residue. Preferred is leuprolide acetate (chemical name: 5-oxo-L-prolyl-L-histidyl-L-tryptophyl-L-seryl-L-tyrosyl-D-leucyl-L-leucyl-L-arginyl-N-ethyl-L-prolinamide acetate salt). It is available under the trade name LUPRON® or LUPRON DEPOT®, available from TAP Pharmaceutical Products, Inc., Lake Forest, Ill., United States of America (see, e.g., U.S. Pat. Nos. 4,897,256 and 5,446,025, the disclosures of each of which are incorporated herein by reference).

The composition also includes chitosan. Suitable chitosans include, without limitation, all derivatives of chitin or poly-N-acetyl-D-glucosamine, including all polyglucosamine and oligomers of glucosamine materials of different molecular weights in which a proportion of the N-acetyl groups have been removed through hydrolysis (i.e., deacetylated chitins).

Chitosan, chitosan derivatives or salts of chitosan (such as nitrate, phosphate, sulphate hydrochloride, glutamate, lactate, or acetate salts) are included. By “chitosan derivatives” it is meant ester, ether or other derivatives formed by bonding of acyl and/or alkyl groups with OH groups, but preferably not the NH₂ groups, of chitosan. Examples of chitosan derivatives for use in the invention include, without limitation, O-alkyl ethers of chitosan and O-acyl esters of chitosan. Modified chitosans, particularly those conjugated to polyethylene glycol, are also included as chitosans for use in the invention.

While any chitosan may be used, it is preferred that selected chitosan preferably has a molecular weight of at least 4,000 daltons, preferably a molecular weight of about 25,000 to about 2,000,000 daltons, and most preferably about 50,000 to about 300,000 daltons. Chitosans of varying lower molecular weights may be prepared by enzymatic degradation of chitosan using chitosanase or by the addition of nitrous acid. Both procedures are well known to those skilled in the art and are described in recent publications (Li et al. (1995) Plant Physiol. Biochem. 33, 599-603; Allan and Peyron, (1995) Carbohydrate Research 277, 257-272; Damard and Cartier, (1989) Int. J. Biol. Macromol. 11, 297-302), the contents of each of which are incorporated herein by reference.

Preferably, the chitosan selected for use in the compositions and the methods of the invention is water-soluble. It may be produced from chitin by deacetylation to a degree of greater than about 40%, preferably between about 50% and about 98%, and more preferably between about 70% and about 90%. Suitable chitosan containing formulations which can be used in the methods and compositions of the invention include, for example, those set forth in U.S. Pat. Nos. 6,207,197; 6,342,251; 6,391,318; 6,432,440; 6,465,626; and 6,534,065, the contents of each of which are incorporated herein by reference.

Preferred is a chitosan sold under the tradename PROTASAN®, available from NovaMatrix, FMC BioPolymer, Drammen, Norway. Additionally, other low and medium viscosity chitosans may be obtained from various sources, including Seigagaku America Inc., Maryland, United States of America; Meron Biopolymers, Kerala, India; Vanson Ltd., Virginia, United States of America; and AMS Biotechnology Ltd., Abingdon, United Kingdom. Suitable chitosan derivatives for use in the composition in the methods of the invention include those that are disclosed in Roberts, Chitin Chemistry, MacMillan Press Ltd., London (1992), the disclosure of which is incorporated herein by reference.

When preparing the composition, the amount of leuprolide present in the composition will vary depending on various factors well known to persons of skill in the art, including the disease or disorder for which the composition is intended to treat, the chemical nature of the selected leuprolide and/or of the overall composition, the gender and other characteristics of the patients the intended dosage regime, etc. However, it is generally preferred that the amount of leuprolide is present in the composition in a concentration of about 5 mg/ml to about 50 mg/ml, about 10 mg/ml to about 40 mg/ml or about 20 mg/ml to about 35 mg/ml. Similarly, the amount of chitosan present in the solution may vary. Preferred are sufficient amounts such that the ratio of the leuprolide to chitosan (by weight) is about 10 parts leuprolide to about 1 part chitosan, 5 parts leuprolide to about 1 part chitosan, or 2 parts leuprolide to about 1 part chitosan.

The leuprolide containing composition is administered by contacting the composition to any mucosal surface (or a non-keratinized epithelial surface) of a mammal, preferably excluding the mucosa of the gastrointestinal tract. Such surfaces include nasal mucosa, buccal mucosa, vaginal mucosa, rectal mucosa, an eye, and pulmonary mucosa. Most preferred is the nasal mucosa.

A vehicle may be included in the composition. The composition is preferably formulated for administration via the nasal route (i.e., by contacting it to the nasal mucosa), and therefore may contain a nasal administration vehicle. The nasal administration vehicle may be any that is pharmaceutically acceptable for such purpose, and may take any suitable form, including a powder, liquid or semi-liquid preparations, such as liniments, lotions, oil-in-water or water-in-oil emulsions, such as creams, ointments or pastes, and solutions or suspensions. Preferred nasal administration vehicles are water, saline, and other aqueous solutions.

If the nasal administration vehicle is in the form of a powder, it is desirable that the overall composition (including the leuprolide) has an average particle from about 0.2 to 500 micrometers. Powder nasal administration vehicles may include, for example, sugars, amino acids, cellulose polymers, cyclodextrins, and solid polyethylene glycols. Specific examples include lactose, sucrose, glucose, trehalose, fructose, glycine, leucine, isoleucine, methylcellulose, and hydroxypropyl methylcellulose.

Such composition can be administered nasally in the manner in which snuff is taken, i.e., by rapid inhalation through the nasal passage from a container of the powder held close to the nose. Alternatively the powder may be atomized and delivered to the nasal cavity. This may be accomplished by, e.g., use of delivery devices of a type where energy from patient inhalation (sniffing) is used to aerosolize the powder into the nasal cavity or where the device itself provides the aerosolization energy, such as via compressed air. An example of the former device is manufactured by Pfeiffer GmbH, Radolfzell, Germany, and an example of the latter is the “monopowder” available from Valois SA, Marly-le-Roi, France.

As will be recognized by one of skill in the art, the precise nature and combination of amounts of ingredients of the formulation will vary depending on numerous factors, including the chemical nature of the chitosan and/or leuprolide selected, the physiology of the patient to which the composition is to be administered, the dosage, and the illness or disorder for which the therapy is being provided. However, the formulation may include other pharmaceutically acceptable additives, such as preservatives (such as benzalkonium chloride or methylhydroxybenzoate), non-ionic or ionic surfactants, antibiotics, anti-inflammatory agents, salts, vitamins, flavoring agent (such as saccharin sodium), volatile oils, buffering agents, and/or surface active agents. A preferred formulation does not contain sorbital. The finished composition may be buffered to a suitable pH as is known in the art, if necessary.

Although any additive or combination of additives may be included, it is preferred that the composition contains solely one bioadhesive material, chitosan, and that preferably no other bioadhesive materials (such as gelatins, agar, etc.) are included. It is hypothesized that interaction of the positive charges present on the chitosan molecules with the negatively charged sialic acid residue of mucin (present in mucus) makes the chitosan highly suitable and that additional other bioadhesives may interfere with these interactions. For similar reasons, it may be preferable to omit any additional emulsifiers from the compositions.

The composition can be provided with an applicator, device, or dispenser for achieving application to the desired mucosal surface. For example, if the composition is intended to be administered nasally, the applicator may be a sponge- or brush-tipped wand or an atomizer or mister or such other devices that provide an atomized or aerosolized spray. For example, use of a spray device may be preferred. Spray devices can be a single (“unit”) dose or multiple dose systems, for example systems including a bottle, pump and actuator, and are available from various commercial sources, including Pfeiffer GmbH, Radolfzell, Germany; Valois SA, Marly-le-Roi, France, Sainte Gobain Calmar, City of Industry, Calif., United States of America; and Becton, Dickinson and Company, Franklin Lakes, N.J., United States of America. Electrostatic spray devices, such as those described in U.S. Pat. No. 5,655,517 (the contents of which are incorporated herein by reference), are also suitable for the intranasal administration of the compositions of the invention. If the composition is to be administered via other mucosal routes, the applicator may be, for example, a suppository, an eyedropper, a tampon, an ear syringe, or a metered dose inhaler.

The compositions of the invention may be used to accomplish transmucosal, more specifically nasal administration of leuprolide for the treatment of all diseases, conditions and disorders known or later understood in the art to be effectively treated, interdicted, or ameliorated by it (“leuprolide modulated conditions”). Such diseases, conditions, and disorders may include prostate cancers, arthrosclerosis, impotence, endometriosis, uterine fibroid tumors, precocious puberty, uterine leiomyomas, hypogonadism, premenstrual syndrome, breast cancers, and ovarian and uterine cancers. In addition, reproductive therapy (assisted reproduction) or regulation of hormonal cycles, such as the increase or reduction of testosterone or estrogen production or control of reproductive capacity may also be accomplished using the leuprolide compositions and methods of the invention.

The methods and compositions of the invention may be applied to any mammalian organism, such as livestock (cows, pigs, and sheep), horses, cats, dogs, mice, rats, etc. Application to humans is preferred.

For example, the methods and composition of the invention can be used in the method of administering leuprolide to a mammal (preferably a human) suffering from one or more of the leuprolide modulated conditions by contacting the nasal mucosa of the patient once, or more than once, over a period of time. The period of time may be one to fourteen days, greater than fourteen days, fifteen to twenty-eight days, or greater than one month. The methods include inhibition of the net production of testosterone in a male mammal. Such method includes repeatedly contacting the nasal mucosa of a mammal with the composition of the invention, over a time period, such as those listed above. Alternatively, the method may be a method of treating endometriosis in a female mammal or inhibiting the net production of estrogen in a female mammal, by administering the composition over, a period of time at repeated dosages.

### EXAMPLE 1

A composition for the intranasal delivery of leuprolide was prepared by combining the following components (Table 1):

TABLE 1
Leuprolide acetate nasal solution, 10 mg/ml

| | |
|---|---|
| leuprolide acetate | 10.0 mg/mL |
| chitosan glutamate (PROTASAN ®) | 5.0 mg/mL |
| benzalkonium chloride NF | 0.2 mg/mL |
| Sodium chloride NF | 7.5 mg/mL |
| water for injection USP | q.s. to volume |

The chitosan glutamate was a pharmaceutical grade chitosan sold under the trade name PROTASAN®, available from NovaMatrix/FMC BioPolymer, Drammen, Norway.

### EXAMPLE 2

A composition for the intranasal delivery of leuprolide was prepared by combining the following as shown in Table 2:

TABLE 2
Leuprolide acetate nasal solution, 20 mg/ml

| | |
|---|---|
| leuprolide acetate | 20.0 mg/mL |
| chitosan glutamate (PROTASAN ®) | 5.0 mg/mL |
| benzalkonium chloride NF | 0.2 mg/mL |
| Sodium chloride NF | 7.5 mg/mL |
| water for injection USP | q.s. to volume |

The chitosan glutamate was a pharmaceutical grade chitosan sold under the tradename PROTASAN®, available from NovaMatrix/FMC BioPolymer, Drammen, Norway. This composition was administered as described in Example 3 below.

### EXAMPLE 3

### Intranasal Administration (IN) of Leuprolide to Women

In the following example, the inventors sought to compare the intranasal (“IN”) administration of leuprolide using the methods and compositions of the invention with prior art methods of administration (subcutaneous (“SQ”) and intravenous (“IV”)).

The design of the experiment was a five-way, crossover, randomized, partially double-blind, volunteer trial. Women were given leuprolide 1 mg IV and SQ, and IN as novel formulation at 1 mg (one spray), 2 mg (one spray) and 6 mg (three sprays), in random order. There was one dosing regimen on each of five separate days, with each day separated by at least two washout days. Blood for leuprolide measurement was collected via indwelling catheter at baseline and on eighteen occasions, up to twenty four hours after dosing. Plasma was separated and the leuprolide concentrations determined by enzyme linked immunosorbant assays (ELISA). Throughout the study subjects were monitored for adverse events and given nasal examinations.

Fifteen healthy women (mean age, twenty-seven years) were included in the safety analysis. No serious adverse events were reported, but some of the subjects noted rhinorrhea and altered taste sensation at some time during the trial, usually after IN dosing; these events were mild and transient in nature. Pharmacokinetics were evaluated in twelve subjects that completed the study and selected values ±SD are shown in Table 3.

TABLE 3

| Parameter | IV, I mg | SQ, 1 mg | IN, 1 mg | IN, 2 mg | IN, 6 mg |
|---|---|---|---|---|---|
| Max plasma | 85 ± 33 | 35 ± 8 | 5 ± 3 | 11 ± 6 | 22 ± 11 |
| conc, ng/mL | | | | | |
| Area under | 120 ± 25 | 111 ± 21 | 15 ± 8 | 31 ± 16 | 56 ± 28 |
| curve, | | | | | |
| ng*hr/mL | | | | | |
| Absolute | 100 | 95 ± 17 | 13 ± 7 | 14 ± 7 | 8 ± 3 |
| bioavail- | | | | | |
| ability, % of | | | | | |
| IV | | | | | |

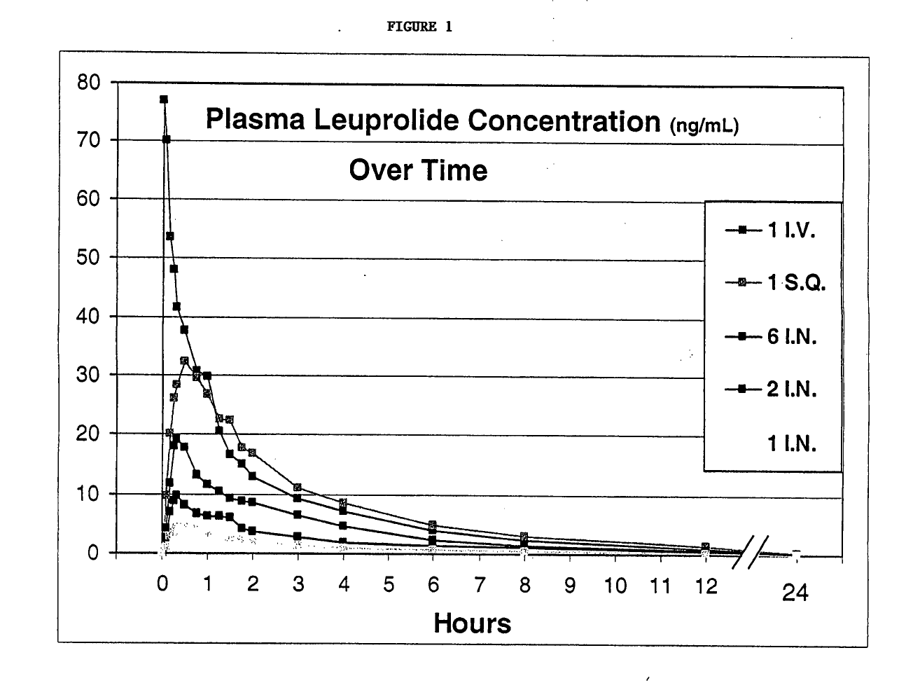

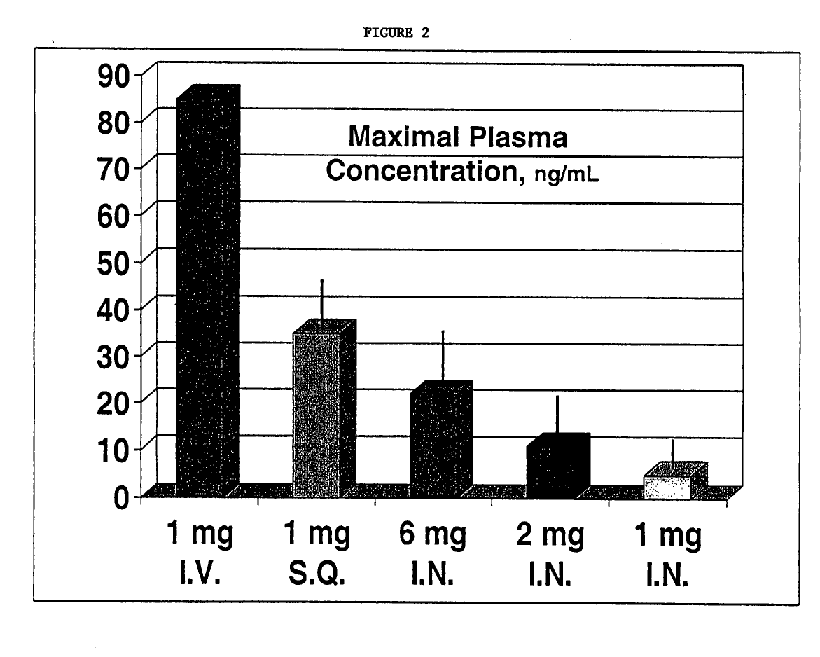

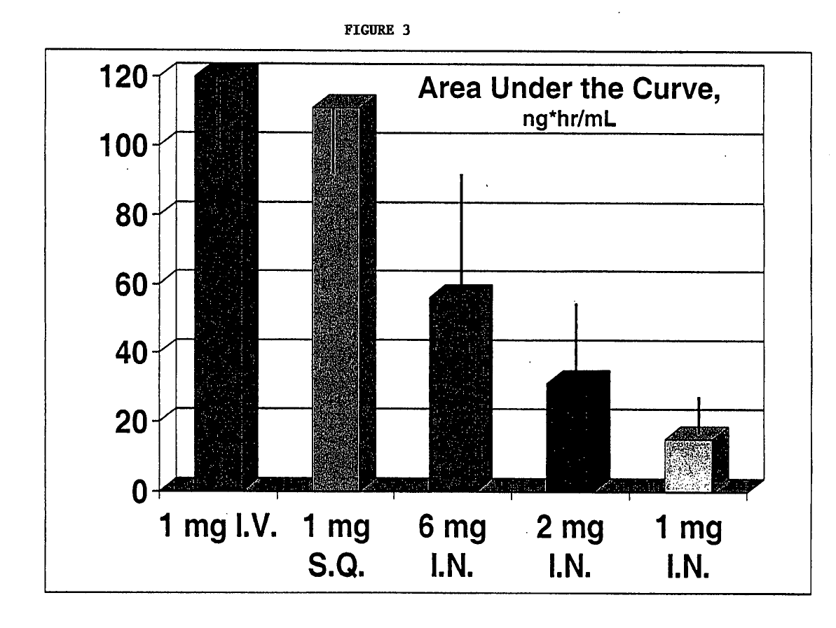

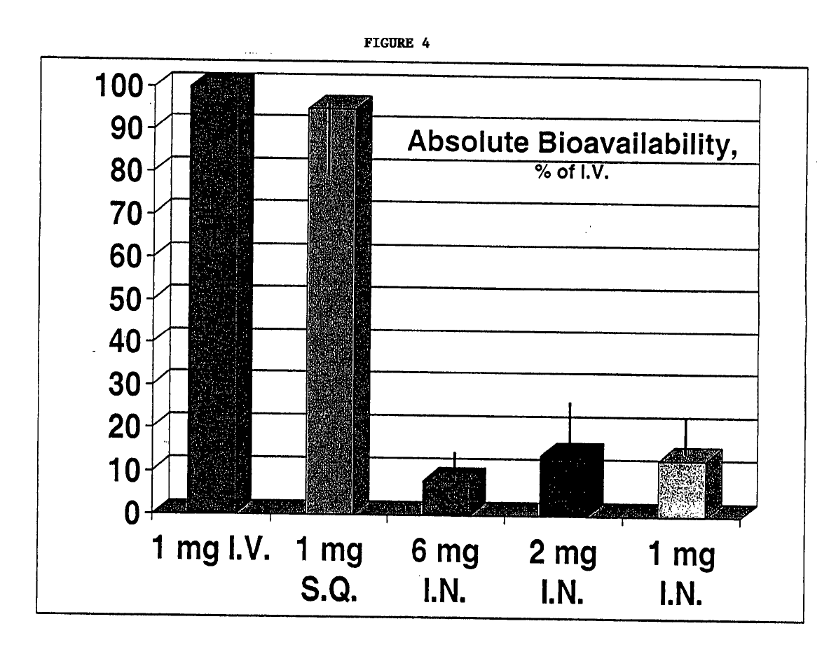

These results are graphically illustrated in FIGS. 1-4. FIG. 1 shows the plasma concentration (ng/ml) of leuprolide of each of the subjects plotted over time. FIG. 2 is a bar graph illustrating the maximal plasma concentration achieved by each method of administration. FIG. 3 shows the area under the curve (AUC) of concentration versus time for each method of administration. FIG. 4 shows the absolute bioavailability of leuprolide by method of administration as a percent value of the bioavailability of intravenously administered leuprolide. Thus, it can be seen that the intranasally administered leuprolide composition of the invention was well tolerated, and well absorbed, and produced plasma leuprolide levels which approximate those seen following traditional SQ administration.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A composition for nasal administration comprising:
a) leuprolide, and
b) one bioadhesive material that is chitosan.

2. The composition of claim 1, further comprising a nasal administration vehicle.

3. The composition of claim 1, wherein the chitosan has a molecular weight of at least 4,000 daltons.

4. The composition of claim 1, wherein the chitosan is chitosan glutamate.

5. The composition of claim 1, wherein the leuprolide is leuprolide acetate.

6. The composition of claim 1, wherein the leuprolide is a polypeptide of greater than nine amino acid residues that comprises SEQ ID NO: 1.

7. The composition of claim 1, wherein the leuprolide is SEQ ID NO: 1.

8. The composition of claim 1, wherein the chitosan and the leuprolide are present in a ratio of about 10 parts by weight leuprolide to about 1 part by weight of chitosan.

9. The composition of claim 1, wherein the chitosan and the leuprolide are present in a ratio of about 5 parts by weight leuprolide to about 1 part by weight of chitosan.

10. The composition of claim 1, wherein the chitosan and the leuprolide is present in and ratio of about 2 parts by weight leuprolide to about 1 part by weight of chitosan.

11. A composition for nasal administration consisting essentially of chitosan, leuprolide, a preservative, and a nasal administration vehicle.

12. A method of administration of leuprolide to a mammal suffering from a leuprolide modulated condition comprising contacting the nasal mucosa of a mammal with a composition, wherein the composition comprises one bioadhesive material that is chitosan and a therapeutically effective amount of leuprolide.

13. The method of claim 11, wherein the composition comprises chitosan having a molecular weight of at least 4,000 daltons.

14. The method of claim 12, wherein the chitosan of the composition is chitosan glutamate.

15. The method of claim 12, wherein the leuprolide of the composition is leuprolide acetate.

16. The method of claim 12, wherein the leuprolide present in the composition is a polypeptide of greater than nine amino acid residues that comprises SEQ ID NO: 1.

17. The method of claim 12, wherein the leuprolide present in the composition is SEQ ID NO: 1.

18. The method of claim 12, wherein the chitosan and the leuprolide are present in the composition in a ratio of about 10 parts by weight leuprolide to about 1 part by weight of chitosan.

19. The method of claim 12, wherein the chitosan and the leuprolide are present in the composition in a ratio of about 5 parts by weight leuprolide to about 1 part by weight of chitosan.

20. The method of claim 12, wherein the chitosan and the leuprolide are present in the composition in a ratio of about 2 parts by weight leuprolide to about 1 part by weight of chitosan.

21. The method of claim 9, wherein the mammal is a human.

22. The method of claim 12, wherein the leuprolide modulated condition is selected from the group consisting of prostate cancers, endometriosis, hypogonadism, premenstrual syndrome, uterine leiomyomas, and precocious puberty.

23. A method of inhibiting the net production of estrogen in a female mammal comprising repeatedly contacting the nasal mucosa of the mammal with a composition over a time period, wherein the composition comprises one bioadhesive material that is chitosan and a therapeutically effective amount of leuprolide.

24. The method of claim 23, wherein the nasal mucosa is contacted with the composition over a time period that is at least fourteen days in duration.

25. A method of inhibiting the net production of testosterone in a male mammal comprising repeatedly contacting the nasal mucosa of the mammal with a composition over a time period, wherein the composition comprises chitosan and a therapeutically effective amount of leuprolide.

26. The method of claim 25, wherein the nasal mucosa is contacted with the composition over a time period that is at least fourteen days in duration.

27. A method of treating endometriosis in a female mammal comprising contacting the nasal mucosa of the mammal with a composition wherein the composition comprises one bioadhesive material that is chitosan and a therapeutically effective amount of leuprolide.

28. The method of claim 27, wherein the composition comprises chitosan having a molecular weight of at least 4,000 daltons.

29. The method of claim 27, wherein the composition comprises chitosan glutamate.

30. The method of claim 27, wherein the composition comprises leuprolide acetate.

31. The method of claim 27, wherein the chitosan and the leuprolide are present in the composition in a ratio of about 10 parts by weight leuprolide to about 1 part by weight of chitosan.

32. The method of claim 27, wherein the chitosan and the leuprolide are present in the composition in a ratio of about 5 parts by weight leuprolide to about 1 part by weight of chitosan.

33. The method of claim 27, wherein the chitosan and the leuprolide are present in the composition in a ratio of about 2 parts by weight leuprolide to about 1 part by weight of chitosan.

34. The method of claim 27, wherein the mammal is a human.
